(19) United States
(12) Patent Application Publication
(10) Pub. No.: US 2008/0029103 A1
(43) Pub. Date: Feb. 7, 2008

(54) Infant Support and Mattress and Harness Therefor

(76) Inventors: Itzhak Regev, Tel Aviv (IL); Max Jano, Mazkeret Batya (IL)

Correspondence Address: NATH & ASSOCIATES, 112 South West Street, Alexandria, VA, 22314, US

(21) Appl. No.: 11/596,578

(22) PCT Filed: May 4, 2005
(86) PCT No.: PCT/IL05/00470

§ 371 Date: Dec. 7, 2006

(30) Foreign Application Priority Data
May 16, 2004 (IL) 162012

(51) Int. Cl. A61F 5/37; A47C 16/00; A47C 20/02

(52) U.S. Cl. 128/875; 5/509.1; 5/652; 5/655

(57) ABSTRACT

A support system for a person, typically for an infant or child, includes a novel mattress having a movable part that is pivotable with respect to a stationary part, and a novel harness adapted for stably holding the person. The harness is configured to be releasably secured to movable part of the mattress

### FIELD OF THE INVENTION

This invention relates to mattresses and beds specially for babies, infants, and children, in particular to reclining mattresses, and to harnesses used in conjunction therewith.

### BACKGROUND OF THE INVENTION

Inclining or propping up a child or infant on a bed has been suggested by the medical profession as being advantageous for the child or infant, both when awake and asleep. Such a posture is particularly helpful during times of illness, particularly colds or sinus infections, where the inclination of the body allows the sinuses to drain freely. Further, in some treatments for gastroesophageal reflux (GER), a child or infant is inclined in the prone position to aid the esophageal passage in remaining firm and tight, and gravity helps to keep the ingested food in the digestive tract.

Various techniques are known for placing the infant or child in the inclined position. In one example, pillows are placed underneath the infant to position the same as required. However, pillows are usually very soft and easily distort as the overlying infant sinks into the pillow, with the potential danger of suffocation should the infant's head turn so that the mouth and nose press against the pillow. Furthermore, pillows do not provide adequate static support, and the infant can easily change position with respect to the pillows, in many cases actually rolling off it.

Another technique involves inclining the infant's mattress, and many conventional devices exist for adjusting the angle of the plane of the mattress within the crib, for example as described in U.S. Pat. No. 5,208,925. However, such mattresses are often difficult to position and adjust, and slings or the like need to be used with these devices, the slings being connected to the side rails or walls of the crib.

In U.S. Pat. No. 4,657,005, a harness is provided having a pair of anchor straps that are pined to one end of the mattress such that they anchor the harness to the higher portion of the inclined mattress, and lateral straps are pined to the mattress to prevent lateral movement or roll when the infant is in the mattress. While the harness allows the infant some mobility of the arms and legs, pinning the anchor straps or the lateral straps to the mattress is not straightforward.

In U.S. Pat. No. 4,989,286 (Re 34,763) a band of bedding material slips over the end of the crib mattress, when this is inclined, to provide an anchor to an infant support sling connected thereto via a relatively narrow neck portion. However, the neck portion allows the infant to pivot and swing with respect to the band.

In U.S. Pat. No. 4,862,535 and U.S. Pat. No. 5,439,008, problems with inclining the mattress are circumvented by providing a wedge-shaped support member that is placed on the mattress in the crib when needed, and the upper surface of the member comprises strips of material that are used for supporting the infant on the member. When it is desired to have the infant lie in the horizontal position, the wedge-shaped member has to be removed, and suitable storage space found therefor.

### SUMMARY OF THE INVENTION

The present invention provides a support system for enabling an infant to be inclined on a surface at a desired angle, and comprises a mattress configured to have a portion thereof pivotably movable with respect to another, stationary portion of the mattress, and an infant harness that is configured to securely support the infant to the movable part. The mattress and harness are each per se novel.

Thus, the present invention is directed to a support system for an infant or child comprising:
a mattress having a movable part that is pivotable with respect to a stationary part; and
a harness adapted for holding said infant or child, and configured to be releasably secured to said movable part.

The present invention is also directed to an inclinable mattress, comprising an external body of resilient cushioning material and an internally disposed support structure, wherein the support structure is adapted for pivotably moving longitudinally opposed portions of the mattress between at least two angular positions.

The present invention is also directed to a harness for supporting an individual on an inclined mattress or the like, comprising:
a hood comprising a band member and an end member and adapted for being positioned over one end of said mattress when in use; and
a sling adapted for supporting a said individual when in use, wherein said sling is mounted in overlying relationship with said band member.

While the term “infant” and “child” are used herein interchangeably to refer to human beings of age ranging from very young, including newborn babies, to at least young adult, it also refers to any human being irrespective of age, though in particular those having a height that is within the normal height range between newborn and young adult.

The mattress comprises an external body of resilient cushioning material and an internally disposed support structure, wherein the support structure is adapted for pivotably moving longitudinally opposed portions of the mattress between at least two angular positions. The movable part comprises one said longitudinally opposed portion and said stationary part comprises another one of said longitudinally opposed portions.

The support structure comprises a first frame member and a second frame member, said first frame member supporting said movable portion and said second frame member supporting said stationary portion, wherein said first frame member is pivotably mounted with respect to said second frame member. In one embodiment, the first frame member and the second frame member are each substantially rectangular frames, typically U-shaped tubular frames. The first frame is pivotably mounted with respect to said second frame by means of hinges. In one embodiment, the hinges are adapted to enable a range of discrete angles to be provided between said first frame and said second frame, and the range of discrete angles includes at least one of 0°, about 14°, about 28°, or about 43°, or at least one of 0°, about 10°, about 20°, about 30°, about 40° or higher than 40°, or any other specific desired angle. Alternatively, the hinges are adapted to enable any angle within a range of angles to be provided between said first frame and said second frame, and may include, for example, any angle from about 0° to about 45°.

In one embodiment, the body of resilient cushioning material comprises a first layer and a second layer of resilient cushioning material, wherein the said first layer overlies said second layer and is spaced therefrom to accommodate said support structure therebetween. Preferably, end spacers are provided connecting opposed ends of said first and second layers. Typically, the first and second layers and the spacers are integrally formed, and are comprised of a polyurethane foam, preferably of a fire retardant and hypoallergic polyurethane foam.

Optionally, a third layer is overlaid on said first layer, and typically, the third layer is made from polyethylene foam. Further optionally, a cover is provided for covering said mattress in an enveloping manner.

Alternatively, the body of resilient cushioning material comprises inflatable air-filled or liquid-filled substantially rectangular bags, having a space adapted for accommodating the support structure.

Such a mattress can be used on any bed frame of appropriate size and adjusted to the desired angle without difficulty. Indeed, the stationary part may provide sufficient base support so that the mattress can actually be placed on bed springs, a mattress or a flat surface such as the floor, and where necessary the weight of the infant may be counterbalanced by placing an object of suitable weight on the stationary part, for example. When it is required to have the infant lying in the horizontal position, the movable part is merely lowered again, optionally while the infant is still lying on the mattress. This arrangement also allows the user to test for the optimal angle while the infant is harnessed to the mattress, and avoids having to remove the infant every time the mattress angle is re-adjusted.

The mattress can be of a size suitable to enable a child to sleep thereon, and it is thus possible to use a single mattress for a number of different users from infants to children and/or indeed for any user from infancy up until the user grows too tall to fit on the mattress. Alternatively, the mattress may be of a size for an adult to sleep thereon, and it is thus possible to use a single mattress for a number of different users from infants to adults and/or indeed for any user from infancy up until adulthood or until the user grows too tall to fit on the mattress.

The harness comprises:
a hood comprising a band member and an end member and adapted for being positioned over one end of said mattress when in use; and
a sling adapted for supporting a said infant or child when in use, wherein said sling is mounted in overlying relationship with said band member.

At least a majority of the sling is securely mounted in overlying relationship with a lower portion of said band member, and the end member is connected to an upper portion of said band member. Advantageously, the band member of the hood has a portal or cut-out section intermediate the sling and the end member, i.e., in registry with the expected position of head of the infant that is to be supported by the harness, so that the infant can rest its head directly over the mattress rather than over the hood material itself.

The sling comprises a first sling portion joined to a second sling portion via a crutch portion, wherein said second sling portion when in use is folded over said first sling portion via said crotch portion. The sling is preferably in the form of relatively stiff shell-like portions connected together via the crutch portion, and the shells fold at the crutch portion to provide a cavity for securely receiving the infant. One of the shells is removably or permanently secured to the hood at a number of spaced locations, such that at least a majority of the shell overlies the hood, and such a configuration prevent the harness from pivoting or swinging with respect to the hood.

In one embodiment, the sling comprises a first set of suitable fastener means secured to transversely opposed ends of said first sling portion, and a second set of suitable fastener means secured to transversely opposed ends of the second sling portion, wherein when the second sling portion is folded into overlying relationship with the first sling portion, the first and second sets of fastener means, are brought together enabling the said first and second sling portions to be releasably secured to each other. Preferably, the fastener means of each said set preferably comprises a suitable quick release type fastener, including, for example, any one of hook and loop type fasteners, snap type fasteners, adhesive fasteners, and the like. Alternatively, the fastener means may include any one of zips, buttons, snaps, buckles and the like. Preferably, each set of fastener means are adapted to permit attachment of the second sling portion with respect to the first sling portion at a range of different relative positions to accommodate infants of different sizes.

The hood is configured to fit over the end of the movable part of the mattress, and thus can be fitted or removed from the mattress in a very simple manner. The hood is preferably made from a plastic or nylon material, so that the hood can be easily cleaned in situ without having to remove it from the mattress.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a partial isometric view of an embodiment of the system according to the present invention, showing an infant in a face-down position.

FIG. 2 is a partial isometric view of the embodiment of FIG. 1, showing an infant in a face-up position.

FIG. 3 is a longitudinal cross-sectional view of a mattress according the embodiment of FIGS. 1 and 2.

FIG. 4 is a transverse side view of some internal components of the mattress of FIG. 3.

FIG. 5 is a plan view of some internal components of the mattress of FIG. 3.

FIG. 6 is a plan view of the support structure of the mattress of the embodiment of FIG. 3.

FIG. 7 is a side view of the support structure of the mattress of the embodiment of FIG. 3.

FIG. 8 is a plan view of the harness of the embodiment of FIGS. 1 to 3.

### DETAILED DESCRIPTION OF THE INVENTION

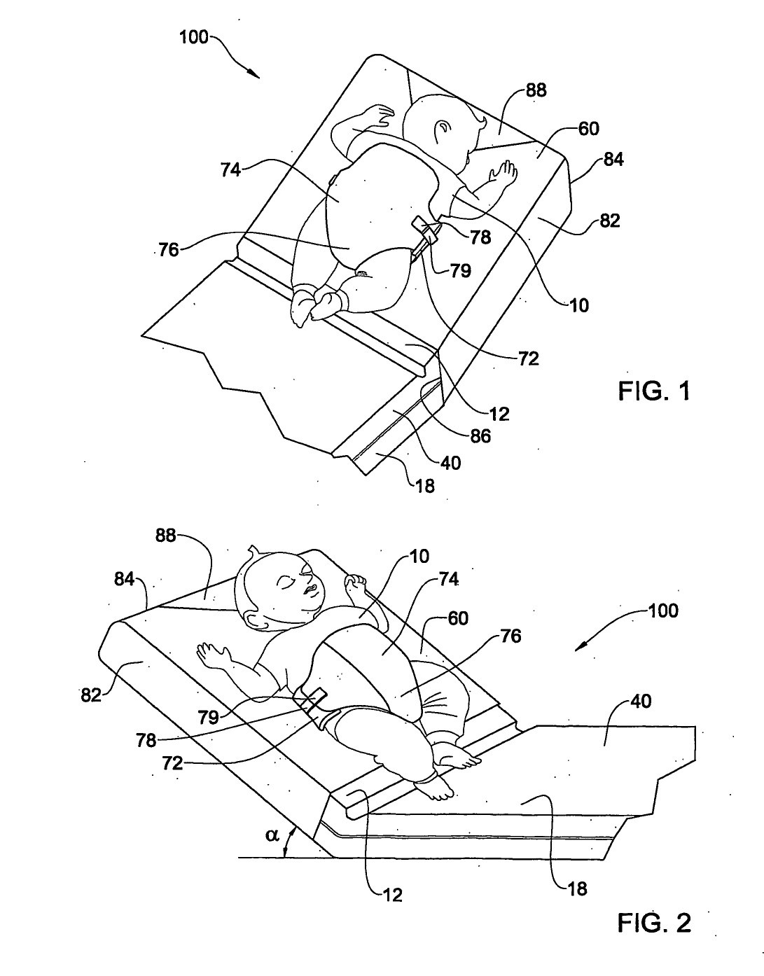

According to the invention, and referring to FIGS. 1 and 2, an inclinable support system, generally designated with the numeral 100, is provided for supporting an infant 10 at any one of a range of angles a to the horizontal. The system 100 comprises an inclinable mattress 40, and a harness 60 that is removably mountable with respect to the mattress 40.

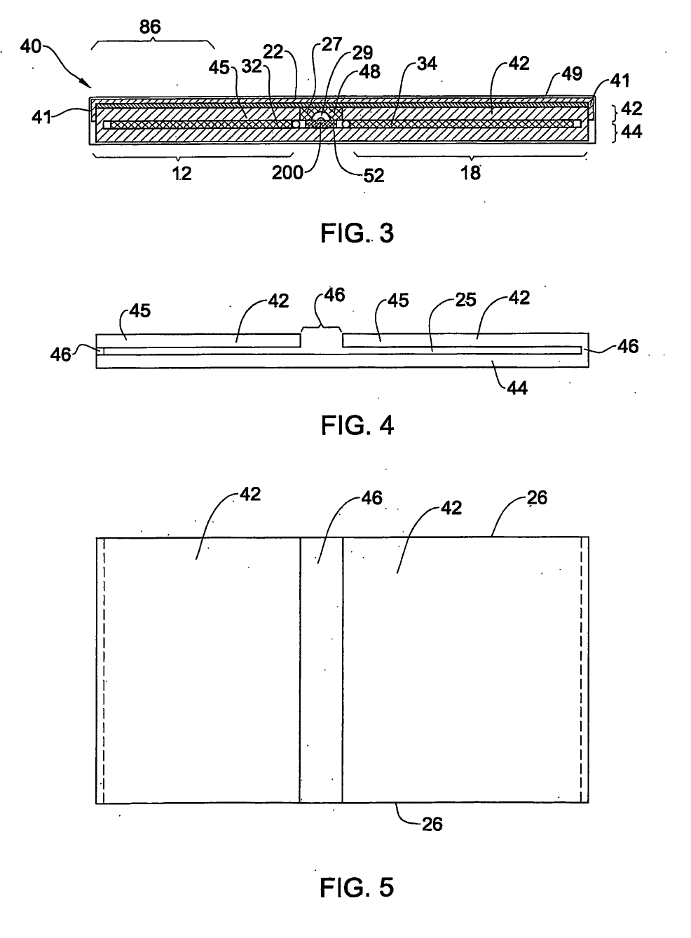

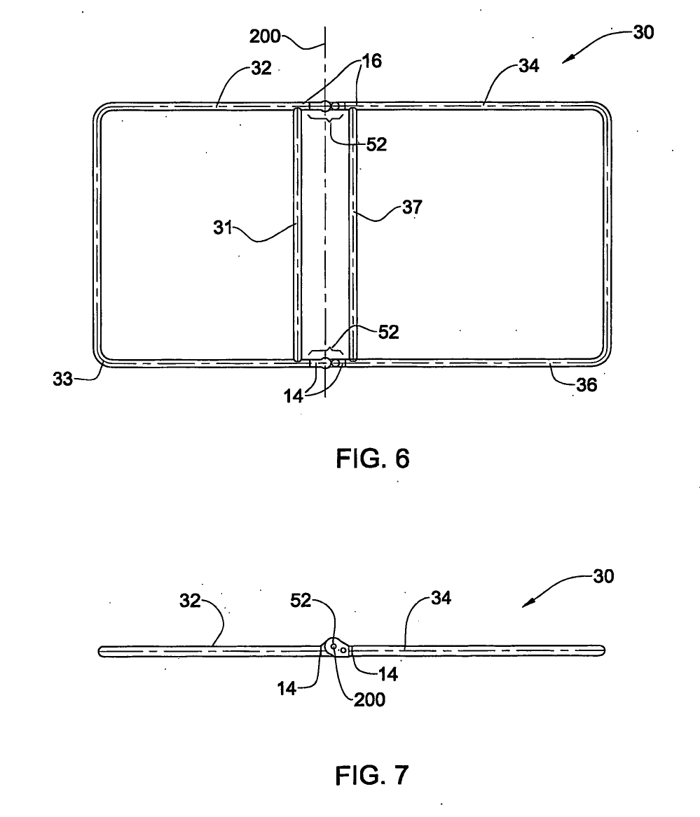

Referring in particular to FIGS. 3 to 7, the inclinable mattress 40, which is novel per se, is in the form of a pad made from a resilient material and having a cavity 25 housing an internal support structure 30. The support structure 30 is thus sandwiched between substantially parallel first layer 42 and second layer 44. The mattress 40 comprises a stationary part or portion 18 and a movable part or portion 12 that is rotatable about a transverse pivot axis 200 with respect to the stationary portion 18 by means of the support structure 30.

In this embodiment, the support structure 30 is in the form of two substantially rectangular frames 32, 34 pivoted together about pivot axis 200. Frame 32 is comprised in the movable portion 12, while frame 34 is comprised in the stationary portion 18. The frames 32, 34, are advantageously formed as U-shaped tubular members 33, 36, respectively, having two pairs of opposed free ends, 14 and 16. At least one of the frames 32, 34 may comprise at least one cross-brace member, 31 and/or 37 respectively. The frames 32, 34 comprise a pivot mechanism comprising a pair of hinges 52, one each connecting corresponding opposed free ends 14, 16, of the members 33, 36.

In this embodiment, each hinge 52 is configured to enable the frames 32, 34 to be set at predetermined discrete angles a to each other, for example, 0°, 14°, 28° 43°; or 0°, 10°, 20°, 30°, 40° or higher; or indeed any set of specific desired angles, and the hinges 52 each comprise a suitable mechanism for this purpose. For example, each hinge 52 may comprise a pair of facing plates, each plate being rigidly connected to a different one of said frames 32, 34. For each pair of plates, the plates are biased to press towards each other, and one plate comprises a notch or a rounded protrusion, for example, while the other plate comprises a series of apertures that are circumferentially spaced along an imaginary circle having its center coaxial with axis 200. The protrusion is configured to fit into each one of the apertures in turn as one frame, and thus one disc, is rotated with respect to the other frame, and thus the other disc, about axis 200. A predetermined torque is required to separate the plates as the protrusion disengages from one aperture and travels towards the next aperture, and thus the frames are stably positioned at mutual angles that are correlated with the position of these apertures. Such arrangements for hinges 52 are known in the art, and indeed there are many other configurations for hinges 52 that are known in the art and capable of providing the required range of discrete angles between the frames 32, 34.

Alternatively, each hinge 52 is configured to enable the frames 32, 34 to be set at any desired angle to each other, for example, in the range 0° to 45° or higher, and the hinges 52 each comprise a suitable mechanism for this purpose. For example, each hinge 52 may comprise a pair of facing plates, each plate being rigidly connected to a different one of said frames 32, 34, and the plates being biased towards each other to provide frictional contact between facing surfaces of the plates. A predetermined torque is required to overcome the static friction between the plates, and when the torque is terminated, the plates, and thus the frames remain stably positioned at the resulting mutual angle. Such arrangements for hinges 52 are also known in the art, and indeed there are many other configurations for hinges 52 that are known in the art and capable of providing the required continuous range of angles between the frames 32, 34.

The mattress 40 comprises a first layer 42 and second layer 44 of a resilient and preferably somewhat compressible padding material, such as for example polyurethane foam, preferably fire retardant and hypoallergic polyurethane foam. This foam may optionally be supplied in a variety of compression densities. The mattress 40 is substantially rectangular in plan configuration, having a length and width typically approximating those of a child, i.e., larger than those of a cot, though in some variations of this embodiment, the width and length of the mattress 40 may approximate that of a cot. In yet other embodiments, the width and length of the mattress 40 may approximate that of an adult-sized bed. The depth of the mattress 40 is typically generally uniform.

The layers 42, 44 are substantially parallel and spaced one from the other to provide cavity 25, and thus serve to sandwich the support structure 30 therebetween. As best shown in FIG. 3 and FIG. 4, the first layer 42 comprises two sections 45 separated by spacing 46. Facing longitudinal ends of the first layer 42 and second layer 44 are connected via spacer strips 46. Preferably, the first layer 42, second layer 44 and strips 46 are integrally formed.

The movable portion 12 thus comprises one of the sections 45, strip 46 and part of second layer 44, and the stationary portion 18 comprises the other one of the sections 45, the other strip 46 and a part of second layer 44. In some embodiments, the mattress 40 may be substantially symmetrical about a vertical plane containing the axis 200 and thus either one of the portions 12, 18 may be considered as a movable portion, and the other one as a stationary portion.

A resilient spacer 27 is provided in space 46, and typically comprises a suitable recess for accommodating the hinges 52 therein and allowing free operation thereof Preferably, the spacer 27 comprises a transverse arched recess 29 extending from one lateral side 26 of the mattress to the other lateral side 26. Typically, spacer 27 is more flexible than the first layer 42, particularly due to the thinner transverse cross-section of the spacer 27 at the apex of the arch thereof, and thus facilitates the bending of mattress 40 about axis 200.

The mattress 40 optionally further comprises a third layer 48 overlying the first layer 42, and typically made from a resilient, though harder material than the first layer 42 or second layer 44, such as for example polyethylene foam.

Optionally, a fourth layer 49 may be provided overlying layer 48, and having wings 41 extending over the longitudinal ends of the mattress 40. The fourth layer 49 is typically made from acrylan and is about 1 cm thick, to provide added comfort to the user. Optionally, the fourth layer 49 may be provided to over the outside of the second layer 44, instead of or in addition to the first layer 42.

Further optionally, the mattress 40 may be fitted with a cover 22, which may be made from any suitable material, including a textile material, or alternatively a wipable material such as for example nylon or the like. Advantageously, the cover 22 completely envelopes the mattress 40 and comprises a zip or other fastener that enables the cover 22 to be selectively removed when desired.

Optionally, the mattress 40 may be covered with a form fitted bed sheet or other covering.

In one non-limiting example of this embodiment, the second layer 44 is about 1.26 m long, about 63 cm wide, and about 3 cm thick. Sections 45 of the first layer each are about 52 cm long, about 63 cm wide, and about 3 cm thick. Strips 46 are about 1.5 cm thick, provide a spacing of about 2 cm between the first and second layers, and are about 63 cm wide. The third and fourth layers, 48, 49 may be approximately 1 cm thick, and about 1.27 m long and 63 cm wide.

In other embodiments not illustrated herein, the mattress may comprise all the elements as described above, mutatis mutandis, but wherein the layers 42, 44, are replaced with one or more air-filled or liquid-filled substantially rectangular bags, similar in shape and dimensions to the layers 42, 44, and having a space adapted for accommodating the support structure 30.

In other embodiments, the support structure 30 may be configured to enable the mattress 40 to pivot in both directions from the horizontal plane. This allows the mattress to be inclined when face up, and also face down, i.e., when the bottom face is turned over so that this is now facing up.

Referring to FIGS. 1, 2, 3 and 8, the harness 60, which is novel per se, comprises a sling 70 mounted onto a hood 80. The hood 80 comprises a band portion 82 and an end portion 84 which together define a cavity into which an end portion 86 of the movable part 12 of mattress 40 is received as the hood 80 is fitted over this end portion 86. The end portion 86 is comprised on the movable part 12 and includes at least a part of one of the sections 45, corresponding portions of the second layer 44, third layer 48, and where appropriate also corresponding portions of fourth layer 49 and cover 22. The hood 80 is made typically from sheet material, preferably a wipable material such as nylon for example, which enables the hood to be kept clean without the need for removing it from the mattress. Alternatively, the hood 80 may be made from a fabric or pulp material. Alternatively, the hood 80 may be made from a disposable material, and thus hood 80 is removable from the sling 60 when desired or soiled, to be replaced by a new hood 80.

Optionally, a head rest (not shown) may be provided on the harness for stabilizing the head of an infant.

Optionally, the hood 80 comprises an opening or portal 88 that enables the infant's head to rest directly against the mattress material when the hood 80 is engaged over the movable part 12. Additionally or alternatively, the hood may be adapted to enable a wipe, towel or the like to be secured to the hood such that the infant's head rests on the wipe or towel rather than directly on the hood.

The sling 70 is configured to encircle and support the torso of the infant, while allowing free movement of the arms, legs and head thereof. The sling 70 comprises a relatively stiff though foldable sheet material and includes a first portion 72, and a second portion 74 which in use is folded over the first portion 72 via a crotch portion 76. The first sling portion 72 and the second sling portion 74 may be shell-shaped, having cavities which are generally complementary to the rounded shape of infant's torso. A first set of suitable fastener means 78 are secured to transversely opposed ends of the first portion 72, and a second set of suitable fastener means 79 are secured to transversely opposed ends of the second portion 74. When the second portion 74 is folded into overlying relationship with the first portion 72, the first and second sets of fastener means, 78, 79 are brought together, enabling the sling portions 72, 74 to be releasably secured to each other. The fastener means 78, 79 of each set preferably comprise any suitable quick release type fastener, such as for example hook and loop (e.g., Velcro) type fasteners, snap type fasteners, adhesive fasteners, and so on. Alternatively, other types of fasteners may be used, including for example, zips, buttons, buckles and so on. Preferably, each set of fastener means 78, 79 permit attachment of the second sling portion 74 with respect to the first sling portion 72 at a range of different relative positions to accommodate infants of different sizes.

The sling material is preferably a washable or otherwise cleanable material, preferably comprising a lining of water resistant fabric or material for enabling the infant to be cleaned up with some ease.

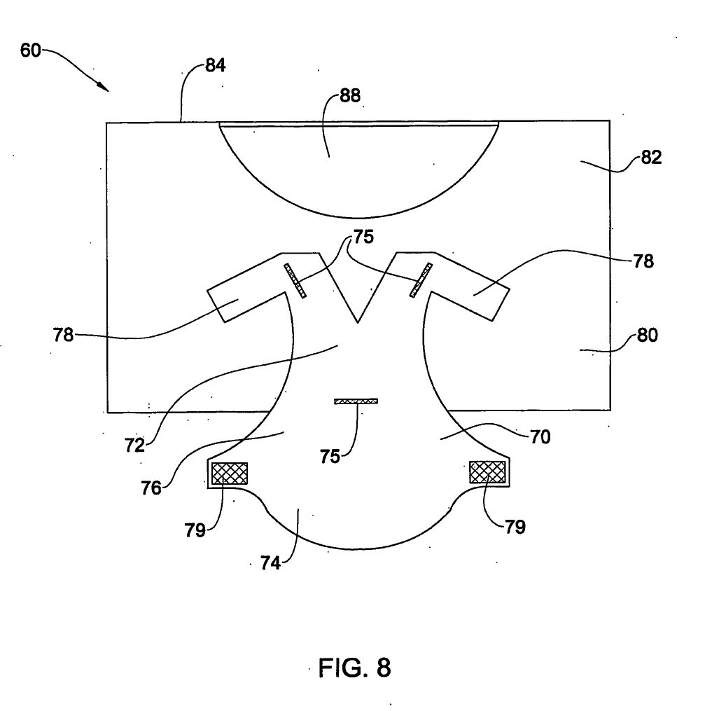

The sling 70 is mounted to the lower part of the hood 80 in such a manner such that when the second sling portion 74 is folded over the first sling portion 72, the second sling portion 74 overlies the lower part of hood 80. The first portion 72 may be permanently joined to the hood 80 via stitching 75, or alternatively releasably joined using any suitable fasteners, including buckles, Velcro, buttons, adhesive strips, zips and so on. In any case, the locations at which the first portion 72 is secured to the hood are such, for example near the crotch part and at the upper extremities, as illustrated in FIG. 8, that at least a majority of the first part 72 is in overlying and secured relationship with respect to the hood 80. This provides stability to the infant in the sling 70, which is then restricted in swinging or pivoting movements with respect to the hood 80.

As illustrated in FIG. 1, the infant's chest and abdomen may be placed against the first sling portion 72, and the infant's back against the second sling portion 74, so that the infant is face-up. Alternatively, and as illustrated in FIG. 2, the infant's chest and abdomen may be placed against the second sling portion 74, and the infant's back against the first sling portion 72, so that the infant is face-down.

In other embodiments, the sling 70 may comprises a different arrangement for securing the infant to the hood 80. For example, the sling may comprise a seat having apertures for the legs of the infant to be inserted therethrough, and suitable straps or the like to restrain the infant on the seat. Such a seat is securely fastened, permanently or removably, to the hood.

The system 100 may be operated in a number of different ways. In one exemplary way, the mattress 40 is placed over bed springs or a box spring of a bed, and the stationary part 18 is secured on the bed by any suitable means, including fasteners, belts, hook-and-loop type fasteners and so on, or, a suitable weight is placed on the stationary part 18 to counterbalance the weight of the infant. Alternatively, the mattress 40 is placed on a regular mattress which is on the bed, or directly on the floor, for example, and the counterbalance weight is placed on the stationary part 18. Then, the movable part 12 is rotated with respect to the stationary part 18 to the desired inclination angle α via hinges 52. The hood 80 is placed over the end 86 of the movable part 12 so that the sling 70 is on the upper portion of the movable part 12. The sling 70 is then opened by unfastening the pairs of fastener means 78, 79, and the second sling portion 74 unfolded with respect to the first sling portion 72. The infant is then held against the first portion 72, face up as in FIG. 1, or face down as in FIG. 2, and the second sling portion 74 is folded about the crutch portion 76 and secured to the first sling portion 72 via the pairs of fastener means 78, 79. Removing the infant is by reversing the above steps. When face down, the infant's face is lying directly on the mattress 40 via portal 88, which is more comfortable than the material of the hood 80, which is typically made from a plastic or nylon-type wipable material. Thus, should the infant experience reflux and vomit, this can be easily removed from the hood 80 without having to dismantle and wash the same. Optionally, a towel or the like may be placed between the infant's head and the hood 80 or movable part 12.

Alternatively, the infant may be secured to the sling 70 while the mattress 40 is horizontal, and the movable part 12 subsequently rotated to the desired angle α. Alternatively, the pairs of fastener means 78, 79 may allow the infant to be inserted into the sling 70 after the first sling portion 72 is secured with the second sling portion. For this purpose, each set of fastener means 78, 79 permit attachment of the second sling portion 74 with respect to the first sling portion 72 at a range of different relative positions, and can be set at a wide position to receive the infant, and then tightened to secure the infant in place.

The mattress 40 comprises a relatively hard layer 48, which is advantageous for an infant or baby up to 9 months old or so, for example. Thereafter, or when there is less need for inclining the mattress 40, the mattress 40 may be turned over so that the bottom thereof, comprising a relatively softer second layer 44, is now face up, affording the infant or child or another user a more comfortable and softer surface.

Thus the mattress of the invention is multi-functional, allowing an infant or child to be inclined in a user friendly manner whenever required or desired, and also allowing the same mattress to be used when the infant is older, or indeed for other users who only require a regular horizontal mattress.

While there has been shown and disclosed exemplary embodiments in accordance with the invention, it will be appreciated that many changes may be made therein without departing from the spirit of the invention.

1. A support system for an infant or child comprising:
a mattress having a movable part that is pivotable with respect to a stationary part; and
a harness adapted for holding said infant or child, and configured to be releasably secured to said movable part.

2. A support system according to claim 1, wherein said mattress comprises an external body of resilient cushioning material and an internally disposed support structure, wherein the support structure is adapted for pivotably moving longitudinally opposed portions of the mattress between at least two angular positions.

3. A support system according to claim 2, wherein said movable part comprises one said longitudinally opposed portion and said stationary part comprises another one of said longitudinally opposed portions.

4. A support system according to claim 2, wherein said support structure comprises a first frame member and a second frame member, said first frame member supporting said movable portion and said second frame member supporting said stationary portion, wherein said first frame member is pivotably mounted with respect to said second frame member.

5. A support system according to claim 4 wherein said first frame member and said second frame member are each substantially rectangular frames.

6. A support system according to claim 4, wherein said first frame is pivotably mounted with respect to said second frame by means of hinges.

7. A support system according to claim 6, wherein said hinges are adapted to enable a range of discrete angles to be provided between said first frame and said second frame.

8. A support system according to claim 7, wherein said range of discrete angles includes at least one of 0°, about 14°, about 28°, or about 43°.

9. A support system according to claim 7, wherein said range of discrete angles includes at least one of 0°, about 10°, about 20°, about 30°, about 40° or higher than 40°, or any other specific desired angle.

10. A support system according to claim 6, wherein said hinges are adapted to enable any angle within a range of angles to be provided between said first frame and said second frame.

11. A support system according to claim 10, wherein said range of angles includes any angle from about 0° to about 45°.

12. A support system according to claim 2, wherein said body of resilient cushioning material comprises a first layer and a second layer of resilient cushioning material, wherein the said first layer overlies said second layer and is spaced therefrom to accommodate said support structure therebetween.

13. A support system according to claim 12, further comprising end spacers connecting opposed ends of said first and second layers.

14. A support system according to claim 13, wherein said first and second layers are comprised of a polyurethane foam.

15. A support system according to claim 14, wherein said first and second layers are comprised of a fire retardant and hypoallergic polyurethane foam.

16. A support system according to claim 12, further comprising a third layer overlaid on said first layer.

17. A support system according to claim 16, wherein said third layer is made from polyethylene foam.

18. A support system according to claim 16, further comprising a cover for covering said mattress in an enveloping manner.

19. A support system according to claim 1, wherein said harness comprises:
a hood comprising a band member and an end member and adapted for being positioned over one end of said mattress when in use; and
a sling adapted for supporting a said infant or child when in use, wherein said sling is mounted in overlying relationship with said band member.

20. A support system according to claim 19, wherein at least a majority of said sling is securely mounted in overlying relationship with a lower portion of said band member, and wherein said end member is connected to an upper portion of said band member.

21. A support system according to claim 19, wherein said band member comprises a portal intermediate said harness and said end member.

22. A support system according to claim 19, wherein said sling comprises a first sling portion joined to a second sling portion via a crutch portion, wherein said second sling portion when in use is folded over said first sling portion via said crotch portion.

23. A support system according to claim 22, further comprising a first set of suitable fastener means secured to transversely opposed ends of said first sling portion, and a second set of suitable fastener means secured to transversely opposed ends of the second sling portion, wherein when the second sling portion is folded into overlying relationship with the first sling portion, the first and second sets of fastener means, are brought together enabling the said first and second sling portions to be releasably secured to each other.

24. A support system according to claim 23, wherein the said fastener means of each said set preferably comprise a suitable quick release type fastener.

25. A support system according to claim 24, wherein said fastener means include any one of hook and loop type fasteners, snap type fasteners, adhesive fasteners, and the like.

26. A support system according to claim 23, wherein said fastener means include any one of zips, buttons, snaps, buckles and the like.

27. A support system according to claim 23, wherein each set of fastener means are adapted to permit attachment of the second sling portion with respect to the first sling portion at a range of different relative positions to accommodate infants of different sizes.

28. An inclinable mattress comprising an external body of resilient cushioning material and an internally disposed support structure, wherein the support structure is adapted for pivotably moving longitudinally opposed portions of the mattress between at least two angular positions.

29. A mattress according to claim 28, wherein said mattress comprises a movable part comprising one said longitudinally opposed portions and a stationary part comprising another one of said longitudinally opposed portions.

30. A mattress according to claim 28, wherein said support structure comprises a first frame member and a second frame member, said first frame member supporting said movable portion and said second frame member supporting said stationary portion, wherein said first frame member is pivotably mounted with respect to said second frame member.

31. A mattress according to claim 30 wherein said first frame member and said second frame member are each substantially rectangular frames.

32. A mattress according to claim 30, wherein said first frame is pivotably mounted with respect to said second frame by means of hinges.

33. A mattress according to claim 32, wherein said hinges are adapted to enable a range of discrete angles to be provided between said first frame and said second frame.

34. A mattress according to claim 33, wherein said range of discrete angles includes at least one of 0°, about 14°, about 28°, or about 43°.

35. A mattress according to claim 33, wherein said range of discrete angles includes at least one of 0°, about 10°, about 20°, about 30°, about 40° or higher than 40°, or any other specific desired angle.

36. A mattress according to claim 32, wherein said hinges are adapted to enable any angle within a range of angles to be provided between said first frame and said second frame.

37. A mattress according to claim 36, wherein said range of angles includes any angle from about 0° to about 45°.

38. A mattress according to claim 28, wherein said body of resilient cushioning material comprises a first layer and a second layer of resilient cushioning material, wherein the said first layer overlies said second layer and is spaced therefrom to accommodate said support structure therebetween.

39. A mattress according to claim 38, further comprising end spacers connecting opposed ends of said first and second layers.

40. A mattress according to claim 38, wherein said first and second layers are comprised of a polyurethane foam.

41. A mattress according to claim 40, wherein said first and second layers are comprised of a fire retardant and hypoallergic polyurethane foam.

42. A mattress according to claim 38, further comprising a third layer overlaid on said first layer.

43. A mattress according to claim 42, wherein said third layer is made from polyethylene foam.

44. A mattress according to claim 38, further comprising a cover for covering said mattress in an enveloping manner.

45. A harness for supporting an individual on an inclined mattress or the like, comprising:
a hood comprising a band member and an end member and adapted for being positioned over one end of said mattress when in use; and
a sling adapted for supporting a said individual when in use, wherein said sling is mounted in overlying relationship with said band member.

46. A harness according to claim 45, wherein at least a majority of said sling is securely mounted in overlying relationship with a lower portion of said band member, and wherein said end member is connected to an upper portion of said band member.

47. A harness according to claim 45, wherein said band member comprises a portal located intermediate said harness and said end member.

48. A harness according to claim 45, wherein said sling comprises a first sling portion joined to a second sling portion via a crutch portion, wherein said second sling portion when in use is folded over said first sling portion via said crotch portion.

49. A harness according to claim 48, further comprising a first set of suitable fastener means secured to transversely opposed ends of said first sling portion, and a second set of suitable fastener means secured to transversely opposed ends of the second sling portion, wherein when the second sling portion is folded into overlying relationship with the first sling portion, the first and second sets of fastener means, are brought together enabling the said first and second sling portions to be releasably secured to each other.

50. A harness according to claim 49, wherein the said fastener means of each said set preferably comprise a suitable quick release type fastener.

51. A harness according to claim 50, wherein said fastener means include any one of hook and loop type fasteners, snap type fasteners, adhesive fasteners, and the like.

52. A harness according to claim 49, wherein said fastener means include any one of zips, buttons, buckles and the like.

53. A harness according to claim 49, wherein each set of fastener means are adapted to permit attachment of the second sling portion with respect to the first sling portion at a range of different relative positions to accommodate infants of different sizes.

54. A harness according to claim 45, wherein said harness is adapted for supporting an infant or child.
